(19) United States
(12) Patent Application Publication
(10) Pub. No.: US 2004/0246562 A1
(43) Pub. Date: Dec. 9, 2004

(54) Passive matrix electrophoretic display driving scheme

(75) Inventors: Jerry Chung, Mountain View, CA (US); Wanheng Wang, Sunnyvale, CA (US); Jack Hou, Fremont, CA (US); Rong-Chang Liang, Cupertino, CA (US)

Correspondence Address: HOWROY SIMON ARNOLD & WHITE, LLP, 2941 FAIRVIEW PARK DRIVE, SUITES 200 & 300, FALLS CHURCH, VA, 22042, US

(73) Assignee: SiPix Imaging, Inc.

(21) Appl. No.: 10/837,239

(22) Filed: Apr. 30, 2004

Related U.S. Application Data
(60) Provisional application No. 60/505,340, filed on May 16, 2003.

(51) Int. Cl. G02B 26/00; G01L 1/20; C07K 1/26; C07K 1/28; C02F 1/469; G01F 1/64; B01J 2/00; C08F 2/58; C25B 7/00; G01L 9/18; C25B 15/00; B01D 61/58; B01D 59/50; B01D 59/42; G01N 27/26; B01D 61/42; B01D 57/02

(52) U.S. Cl. 359/296

(57) ABSTRACT

A system and method are disclosed for mitigating the effect of induced reverse bias in a passive matrix electrophoretic display. An intermediate biasing phase is performed prior to the driving cycle, the biasing conditions of the intermediate biasing phase being selected so as to break into at least two steps the transition from the bias condition present prior to the driving cycle to the bias condition applied during the driving cycle. Interposing a settle phase subsequent to the driving phase for each scanned row and prior to the driving phase of the next row to be scanned is disclosed. Adding a pre-drive phase prior to scanning to mitigate the effect of induced reverse bias is disclosed. Adding an inline resistor between a driver and an electrode with which the driver is associated is disclosed. Also disclosed is displaying an image in an electrophoretic display by first driving an array of electrophoretic cells to a white displayed state and then driving background areas to a background display state. In addition, interposing a balance phase after each row is scanned to restore display elements in non-scanning rows to the same initial state is disclosed.

### CROSS REFERENCE TO RELATED APPLICATIONS

This application claims priority to U.S. Provisional Patent Application No. 60/505,340 entitled IMPROVED PASSIVE MATRIX ELECTROPHORETIC DISPLAY DRIVING SCHEME filed May 16, 2003 which is incorporated herein by reference for all purposes.

### FIELD OF THE INVENTION

The present invention relates generally to electrophoretic displays. More specifically, an improved driving scheme for a passive matrix electrophoretic display is disclosed.

### BACKGROUND OF THE INVENTION

The electrophoretic display (EPD) is a non-emissive device based on the electrophoresis phenomenon of charged pigment particles suspended in a solvent. It was first proposed in 1969. The display usually comprises two plates with electrodes placed opposing each other, separated by using spacers. One of the electrodes is usually transparent. A suspension composed of a colored solvent and charged pigment particles is enclosed between the two plates. When a voltage difference is imposed between the two electrodes, the pigment particles migrate to one side and then either the color of the pigment or the color of the solvent can be seen according to the polarity of the voltage difference.

There are several different types of EPDs. In the partition type EPD (see M. A. Hopper and V. Novotny, IEEE Trans. Electr. Dev., Vol. ED 26, No. 8, pp. 1148-1152 (1979)), there are partitions between the two electrodes for dividing the space into smaller cells in order to prevent undesired movements of particles such as sedimentation. The microcapsule type EPD (as described in U.S. Pat. No. 5,961,804 and U.S. Pat. No. 5,930,026) has a substantially two dimensional arrangement of microcapsules each having therein an electrophoretic composition of a dielectric fluid and a suspension of charged pigment particles that visually contrast with the dielectric solvent. Another type of EPD (see U.S. Pat. No. 3,612,758) has electrophoretic cells that are formed from parallel line reservoirs. The channel-like electrophoretic cells are covered with, and in electrical contact with, transparent conductors. A layer of transparent glass from which side the panel is viewed overlies the transparent conductors.

An improved EPD technology was disclosed in co-pending applications, U.S. Ser. No. 09/518,488, filed on Mar. 3, 2000, U.S. Ser. No. 09/759,212, filed on Jan. 11, 2001, U.S. Ser. No. 09/606,654, filed on Jun. 28, 2000 and U.S. Ser. No. 09/784,972, filed on Feb. 15, 2001, all of which are incorporated herein by reference. The improved EPD comprises closed cells formed from microcups of well-defined shape, size and aspect ratio and filled with charged pigment particles dispersed in a dielectric solvent.

An EPD may be driven by a passive matrix system. For a typical passive matrix system, there are column electrodes on the top side (viewing surface) of the display and row electrodes on the bottom side of the cells (or vice versa). The row electrodes and the column electrodes are perpendicular to each other. However, there are two well-known problems, which are associated with EPDs driven by a passive matrix system: cross talk and cross bias. Cross talk occurs when the particles in a cell are biased by the electric field of a neighboring cell. FIG. 1 provides an example. The bias voltage of the cell A drives the positively charged particles towards the bottom of the cell. Since cell B has no voltage bias, the positively charged particles in cell B are expected to remain at the top of the cell. However, if the two cells, A and B, are close to each other, the top electrode voltage of cell B (30V) and the bottom electrode voltage of cell A (0V) create a cross talk electric field which forces some of the particles in cell B to move downwards. Widening the distance between adjacent cells may eliminate such a problem; but the distance may also reduce the resolution of the display.

The cross talk problem may be lessened if a cell has a significantly high threshold voltage. The threshold voltage, in the context of the present invention, is defined to be the maximum bias voltage that may be applied to a cell without causing movement of particles between two electrodes on opposite sides of the cell. If the cells have a sufficiently high threshold voltage the cross-talk effect is reduced without sacrificing the resolution of the display.

Cross bias is also a well-known problem for a passive matrix display. The voltage applied to a column electrode not only provides the driving bias for the cell on the scanning row, but it also affects the bias across the non-scanning cells on the same column. This undesired bias may force the particles of a non-scanning cell to migrate to the opposite electrode. This undesired particle migration causes visible optical density change and reduces the contrast ratio of the display.

An EPD that addresses the problems of cross talk and cross bias is described in U.S. Patent Application No. 60/322,635 (Attorney Docket No. 26822-0042), entitled, “An Improved Electrophoretic Display with Gating Electrodes,” filed Sep. 12, 2001, which is incorporated herein by reference for all purposes. However, even a display employing a cell that exhibits the threshold effect described in the application referenced in the sentence preceding this one may suffer degradations to image quality and/or display performance if the wrong passive matrix driving scheme is used. This is so because even a cell that exhibits a threshold effect (i.e., which does not experience significant particle migration in non-scanning rows even under cross bias conditions, so long as the cross bias does not exceed a threshold) may experience some degradation of image quality due to cross bias under certain conditions, such as prolonged application of a direct current (DC) voltage below the nominal threshold voltage for the cell, or application of a voltage at or below the threshold voltage under conditions where the initial state of the cell is such that undesired particular migration will occur under cross bias conditions below the nominal threshold voltage. Restated, the true threshold voltage of a cell in a particular instance, or under a particular set of conditions, depends not only on the cell structure and materials but also on such additional factors as the length of time the voltage is to be applied and the initial state of the cell. A cell may exhibit a first threshold Vth=A for a voltage applied for a first period=T and a second, lower threshold Vth=B for a voltage applied for twice as long (i.e., 2 T).

Therefore, there is a need for a passive matrix driving scheme that addresses the issues of cross bias and takes into consideration the variables that can affect the threshold voltage Vth that the EPD cells will exhibit under the particular conditions to which they will be subjected under the driving scheme.

A further problem with passive matrix driven EPDs is the problem of reverse bias. For example, a reverse bias condition may be present when the bias voltage on a particular cell changes rapidly by a large increment or decrement, due to the presence of stored charge in the inherent capacitance of the materials and structures comprising the EPD media layer. For example, in a microcup-based EPD such as described in the above-referenced applications, the sealing and adhesive layer, the electrophoretic dispersion, the microcup, and any other insulative layers or materials each has an inherent capacitance (and resistance) associated with it. These capacitances become charged when a bias voltage is applied to a cell, e.g., to drive it to a different display state, and can cause a reverse bias to be present when the bias voltage is changed. Under certain circumstances, this reverse bias can affect the display quality by causing charged pigment particles in affected cells to migrate away from the position to which they have been driven.

FIG. 2A shows a typical EPD cell 200 comprising a quantity of electrophoretic dispersion, the dispersion comprising a plurality of charged pigment particles 204 dispersed in a colored dielectric solvent 206. The dispersion is contained by a top layer of insulating material 208 and a bottom layer of insulating material 210. In one embodiment, the insulating material may comprise a non-conductive polymer. In the cells described in the above-incorporated co-pending patent application, the insulating layer may comprise a sealing and/or adhesive layer, or the micro-cup structure. The dispersion and associated insulating materials are positioned between an upper electrode 212 and a lower electrode 214.

In FIG. 2A, three points labeled “A”, “B”, and “C” are shown, with point A being located at the top of the insulating layer 208, point B being located at the bottom of insulating layer 208 (i.e., at the top of the dispersion 202), and point C being located at the bottom of insulating layer 210. FIG. 2B shows an equivalent circuit for that portion of the cell 200 of FIG. 2A that lies between points A and C. In FIG. 2B, the capacitor C1 and the resistor R1 represent the inherent capacitance and resistance of the upper insulating layer 208. Likewise, the capacitor C2 and the resistor R2 represent the inherent capacitance and resistance of the lower insulating layer 210. The dispersion 202 likewise would have a capacitance and resistance associated with it.

As illustrated in FIGS. 2A and 2B, if a driving voltage Vd is applied to the upper electrode 212 and the lower electrode 214 is held at ground potential, the voltage applied across the dispersion itself will initially be very near Vd, but will decrease somewhat as the capacitors C1 and C2 are charged. FIG. 3 illustrates this reduction in the voltage applied across the dispersion as the capacitors C1 and C2 are charged, as well as the induced reverse bias effect that may occur if the voltage applied across the cell 200 is changed suddenly by a large increment, such as by transitioning from the driving voltage Vd to zero volts. At point A, the voltage applied would be a square waveform, quickly rising to Vd initially, maintaining that level, and then quickly dropping to and staying at zero (as illustrated by the dashed lines in FIG. 3). However, when the voltage applied at point A is dropped to zero, the dispersion is actually subjected to an induced reverse bias while the capacitances C1 and C2 discharge, which results in a negative field being applied to the dispersion, at least on a transient basis (see the point labeled “Reverse Bias” in FIG. 3). Once the capacitances have discharged, the voltage applied to the dispersion (i.e., at point B) settles back to zero. Depending on the conditions and cell design, the transient induced reverse bias may cause degradation of the image quality, such as by causing charged particles to migrate away from a position to which they have been driven to display a desired image.

A similar problem occurs, as noted above, when a bias voltage lower than the cell threshold voltage is applied without interruption for a prolonged period. Such an uninterrupted voltage is sometimes referred to as a “DC” or “direct current” voltage or component. In such conditions, charged particles may migrate to an undesired position even though the bias voltage is less than the threshold voltage, because the effective threshold voltage is lower for bias voltages applied over a long period.

Therefore, there is a need for driving scheme for a passive matrix EPD in which the problems of reverse bias and undesired effects of DC bias voltages are mitigated.

### BRIEF DESCRIPTION OF THE DRAWINGS

The present invention will be readily understood by the following detailed description in conjunction with the accompanying drawings, wherein like reference numerals designate like structural elements, and in which:

FIG. 1 illustrates the cross talk phenomenon.

FIG. 2A shows a typical EPD cell 200.

FIG. 2B shows an equivalent circuit for that portion of the cell 200 of FIG. 2A that lies between points A and C.

FIG. 3 illustrates the induced reverse bias effect.

FIGS. 4A and 4B-1 through 4B-4 illustrate a 2×2 passive matrix.

FIG. 4C illustrates the “fan in” approach as applied to column electrodes.

FIG. 4D illustrates a connector/adaptor configured to connect an arbitrarily shaped display to a driver IC.

FIG. 5 shows a configuration and scenario used to describe a passive matrix driving scheme used in one embodiment.

FIG. 6 shows a driving scheme for a basic passive matrix EPD.

FIG. 7 shows a passive matrix EPD driving scheme in which an intermediate phase has been added to mitigate reverse bias in non-switching pixels.

FIG. 8 shows a passive matrix EPD driving scheme that further improves on the scheme shown in FIG. 7.

FIG. 9A shows a passive matrix EPD driving scheme in which additional intermediate phases before and after scanning each row have been added to the scheme shown in FIG. 8.

FIG. 9B shows an exemplary driving waveform in which such a pre-drive pulse precedes each scanning cycle.

FIG. 9C illustrates the reduced reverse bias that can be achieved by including a pre-drive phase such as shown in FIG. 9B.

FIG. 10A shows a passive matrix electrophoretic display 1000 on which an image of a circle is to be displayed.

FIG. 10B shows the cells in the background area being driven to the black/background state.

FIG. 11A shows an equivalent circuit 1100 for an EPD cell to which an inline resistor has been added.

FIG. 11B shows a 4×4 array (or portion of an array) in which an inline resistor has been added between the row and column electrodes and their respective drivers.

FIG. 11C shows an alternative arrangement used in one embodiment, in which a switch is provided to enable the inline resistor to be removed from the circuit during driving.

FIG. 12 plots voltage versus time for points A and B of FIG. 11A, which correspond to points A and B in the EPD cell shown in FIG. 2A.

FIG. 13 illustrates the reduction in reverse bias that is achieved by using a shorter pulse width.

FIG. 14A shows an exemplary passive matrix EPD comprising a 3×3 array of EPD cells (or pixels comprising one or more EPD cells) during scanning of the first row R1.

FIG. 14B illustrates a balance phase used in one embodiment to return cells in non-scanning rows to the same initial state for scanning.

FIG. 14C illustrates the scanning of the second row R2.

FIG. 14D illustrates a balance phase used in one embodiment to counteract the effect of the positive cross bias on cells in the non-scanning row R3.

### DETAILED DESCRIPTION

It should be appreciated that the present invention can be implemented in numerous ways, including as a process, an apparatus, a system, or a computer readable medium such as a computer readable storage medium or a computer network wherein program instructions are sent over optical or electronic communication links. It should be noted that the order of the steps of disclosed processes may be altered within the scope of the invention.

A detailed description of one or more preferred embodiments of the invention is provided below along with accompanying figures that illustrate by way of example the principles of the invention. While the invention is described in connection with such embodiments, it should be understood that the invention is not limited to any embodiment. On the contrary, the scope of the invention is limited only by the appended claims and the invention encompasses numerous alternatives, modifications and equivalents. For the purpose of example, numerous specific details are set forth in the following description in order to provide a thorough understanding of the present invention. The present invention may be practiced according to the claims without some or all of these specific details. For the purpose of clarity, technical material that is known in the technical fields related to the invention has not been described in detail so that the present invention is not unnecessarily obscured.

A. Derivation of Voltages to be Applied to Scanning Row, Non-Scanning Row, and Column Electrodes to Address the Problem of Cross Bias

The term “threshold voltage” (Vth), in the context of the present disclosure, is defined as the maximum bias voltage that does not cause the particles in a cell to move between electrodes. The term “driving voltage” (Vd), in the context of the present disclosure, is defined as the bias voltage applied to change the color state of a cell, such as by driving the particles in the cell from an initial position at or near one electrode to an end position at or near the opposite electrode. The driving voltage Vd used in a particular application must be sufficient to cause the color state of the cell to change within the required performance parameters of the application, including as measured by such parameters as the time it takes for the state transition to be completed.

A “scanning” row in a passive matrix display is a row in the display that is currently being updated or refreshed. A “non-scanning” row is a row that is not currently being updated or refreshed. A “positive bias”, in the context of the present disclosure, is defined as a bias that tends to cause positively charged particles to migrate upwards (i.e., lower electrode at higher potential than upper electrode). Thus, a positive bias tends to drive positively charged particles towards the viewing surface, such as to switch a cell to the white or “on” state. A “negative bias”, in the context of the present disclosure, is defined as a bias that tends to cause positively charged particles to migrate downwards (i.e., lower electrode at lower potential than upper electrode).

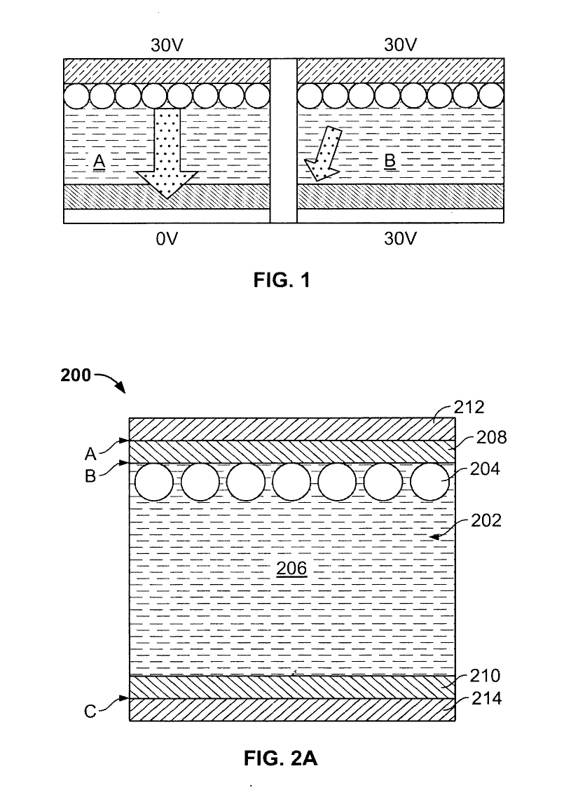

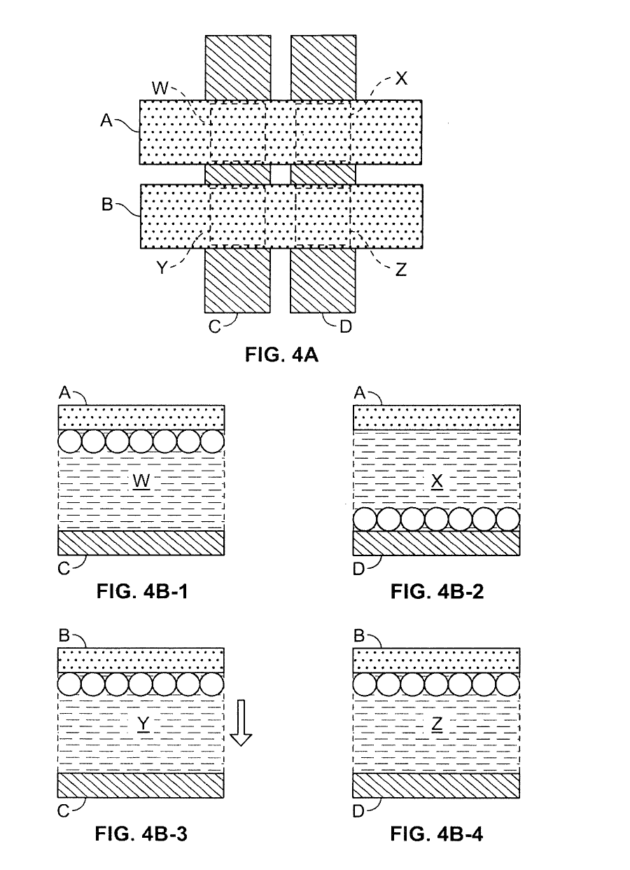

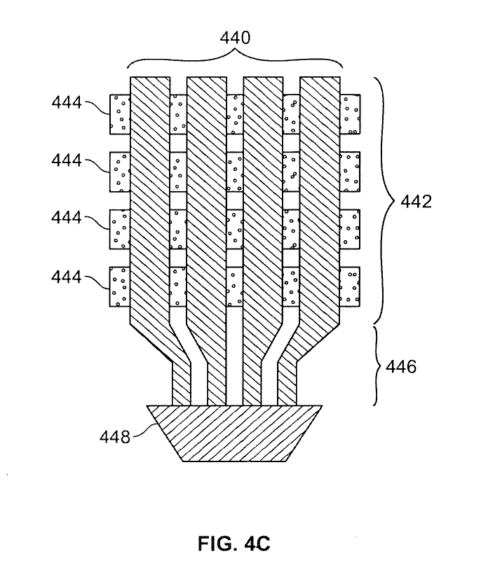

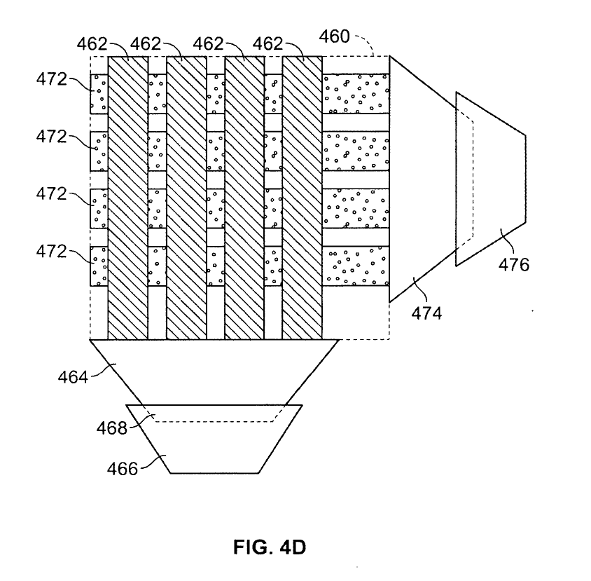

For a typical passive-matrix, the row electrodes may be on the top, and the column electrodes may be on the bottom and perpendicular to the row electrodes, or vice versa. FIGS. 4A and 4B-1 through 4B-4 illustrate a 2×2 passive matrix. FIG. 4A shows the top view of a general 2×2 passive matrix. In this figure, voltage A drives the top, non-scanning row and voltage B drives the bottom, scanning row.

Initially, as shown in FIGS. 4B-1 to 4B-4, the particles in cells W, Y and Z are at the top of the cells, and the particles in cell X are at the bottom of the cell. Assume the scanning row B is to be modified such that the particles in cell Y are moved to the bottom electrode while the particles in cell Z are to be maintained at their current position at the top electrode. The particles in the cells of the non-scanning row should, of course, remain at their initial positions—W at the top electrode and X at the bottom electrode—even if a cross-biasing condition is present.

Because Cells W and X are in a non-scanning row, the goal is to ensure that the particles remain at the current electrode position even when there is a cross bias condition affecting the row. The threshold voltage of the cell is an important factor in these two cases. Unless the threshold voltage is equal to or greater than the cross bias voltage that may be present, the particles in these cells will move when such a cross bias is present, thereby reducing the contrast ratio.

In order to drive the particles in cell Y from the top electrode to the bottom electrode within a specific time period, a driving voltage Vd must be applied. The driving voltage used in a particular application may be determined by a number of factors, including but not necessarily limited to cell geometry, cell design, array design and layout, and the materials and solvents used. In order to move the particles in cell Y without affecting the particles in cells W, X and Z, the driving voltage Vd applied to change the state of cell Y must also be of a magnitude, and applied in such a way, so as not to result in the remaining cells being cross biased in an amount greater than the threshold voltage Vth of the cells.

To determine the minimum threshold voltage needed to avoid unintended state changes in the basic passive matrix illustrated in FIGS. 4A through 4B-4 under these conditions, the following inequality conditions must be satisfied:

A-C<Vth

D-A<Vth

B-C>Vd

B-D<Vth

This system of equations may be solved by summing the three inequalities involving Vth, to yield the inequality (A-C)+(D-A)+(B-D)<Vth+Vth+Vth, which simplifies to B-C<3 Vth, or 3 Vth>B-C. Combining this inequality with the remaining inequality B-C>Vd, we conclude that 3 Vth>B-C>Vd, which yields 3 Vth>Vd or Vth>⅓ Vd. That is, for the passive matrix illustrated in FIGS. 4A through 4B-4, the cells must have a threshold voltage equal to or greater than one third of the driving voltage to be applied to change the state of those cells in which a state change is desired in order to avoid changing as a result of cross bias the state of those cells in which a state change is not desired.

Referring further to FIGS. 4A through 4B-4, if the driving voltage Vd is applied to the scanning row B, then solution of the above inequalities indicates that to ensure that the driving bias voltage is applied to cells to be programmed and that no more than the threshold voltage is applied to other cells (i.e., non-programming cells in the scanning row and all cells in the non-scanning row) the voltage applied to the non-scanning row A should be equal to ⅓ Vd, the voltage applied to the column electrode associated with a cell in the scanning row to be programmed (i.e., display state changed), such as column electrode C, should be 0 volts, and the voltage applied to the column electrode associated with a cell in the scanning row that is not to be programmed (i.e., retain the initial or reset state) should be equal to 2/3 Vd. For example, in one embodiment the driving voltage required to achieve acceptable performance is 30V. If the driving voltage Vd=30V in the passive matrix display illustrated in FIGS. 4A through 4B-4, then the minimum threshold voltage that would be required to retain the initial state of cells W, X, and Z while changing the state of cell Y by applying a driving voltage of 30V to cell Y would be Vth=10V. Assuming B=30V, the solution to the above equations is A=10V, C=0V and D=20V. By reference to FIGS. 4A through 4B-4, one can see that under these conditions the bias applied to each of cells W, X, and Z would in fact be less than or equal to the minimum threshold voltage Vth=10V. It can be shown that this solution applies to a passive matrix of any size, and is not limited to the 2×2 array shown in FIG. 4A.

In one embodiment, a passive matrix electrophoretic display comprises a display media made using a roll-to-roll fabrication process. The display elements comprising the display media comprise microcup-type EPD cells, as described in the patent application incorporated by reference above. The microcups are individually sealed in one embodiment, such that the sheet or roll of display media may be cut to any arbitrary shape. In one embodiment, a connector/adaptor may be provided to connect the row and/or column electrodes of the display media to a driving circuitry, such as a driver integrated circuit (IC). In LCD technology, for example, “fan out” and/or “fan in” approaches are used to connect column and/or row electrodes to a driver IC, the connector (bonding pads) of which typically will not be as wide as the display. FIG. 4C illustrates the fan in an approach as applied to column electrodes. The column electrodes 440 comprise a straight portion 442 overlying the row electrodes 444. The column electrodes further comprise a fan in portion 446, which enables the column electrodes 440 to connect electrically with the driver IC 448. In LCD technology, the approach illustrated in FIG. 4C may be implemented by forming the electrode fan in/fan out portion on the glass substrate of the display.

The above described fan in/fan out approach could be used for a passive matrix EPD, but one would have to know the shape of the display in advance to be able to form the fan in or fan out portion of the electrodes on the substrate. FIG. 4D illustrates an alternative approach, in which a connector/adaptor is provided to enable an arbitrarily shaped display to be connected to a driver IC. In the illustrative example shown in FIG. 4D, a four row by four column section 460 has been cut from a sheet or roll of EPD display media having only straight rows and columns (i.e., no fan in or fan out portions). The column electrodes 462 are connected electrically via a connector/adaptor 464 to the column driver IC 466 by connecting bonding pads associated with the connector/adaptor 464 to corresponding bonding pads associated with the column driver IC 466 in an overlap area 468. In one embodiment, a conductive adhesive, such as ACF or silver paste, is used to bond the column driver IC 466 to the connector/adaptor 464. The connector/adaptor 464 has structures very similar to the fan in portion 446 shown in FIG. 4C. Likewise, the row electrodes 472 are connected via the connector/adaptor 474 to the row driver IC 476. By providing fan in/fan out connectors/adaptors of a variety of shapes and sizes, the full flexibility of an EPD media formed using a roll-to-roll process may be realized by supporting the connection of arbitrarily shaped displays cut from the roll or sheet of display media without requiring changes to or customization of the fabrication process, and without adding complexity and inflexibility to the manufacturing process.

B. Addition of Intermediate, Settling, and/or Pre-Drive Phases to Mitigate Effect of Reverse Bias

The passive matrix driving schemes described in this section assume a passive matrix electrophoretic display comprising an array of electrophoretic cells containing an electrophoretic dispersion including positively charged pigment particles dispersed in a colored dielectric solvent. In one embodiment, the charged pigment particles are white and the dielectric solvent is black or some other contrasting color suitable for use as a background color. In the examples described, the cell threshold voltage Vth is assumed to be 10 V and the cell driving voltage Vd is assumed to be 30 V. In the examples described, the EPD is assumed to comprise an array of column electrodes in an upper layer of the display, above the array of EPD cells, on the viewing surface side of the EPD; and an array of row electrodes in a lower layer of the display, below the array of EPD cells, on the side of the display opposite the viewing surface. In EPDs such as those described, the white pigment particles in cells associated with a pixel would be driven to the viewing surface to display a white color in that pixel and would instead be driven (or caused to remain) at the bottom of the cells to display a black (or other background color) in that pixel (and, in certain embodiments, partly driven to the top or bottom surface, as required, to display a grayscale color in the pixel).

As will be apparent to those of skill in the art, the techniques described herein may be applied as well to other passive matrix EPDs having other types of cells, a different electrophoretic dispersion (e.g., without limitation, one having negatively charged pigment particles), different colors, different electrode arrangements, etc., with readily-calculable changes to the polarity and/or magnitude of the voltages described herein being made, as required, to achieve the results described herein.

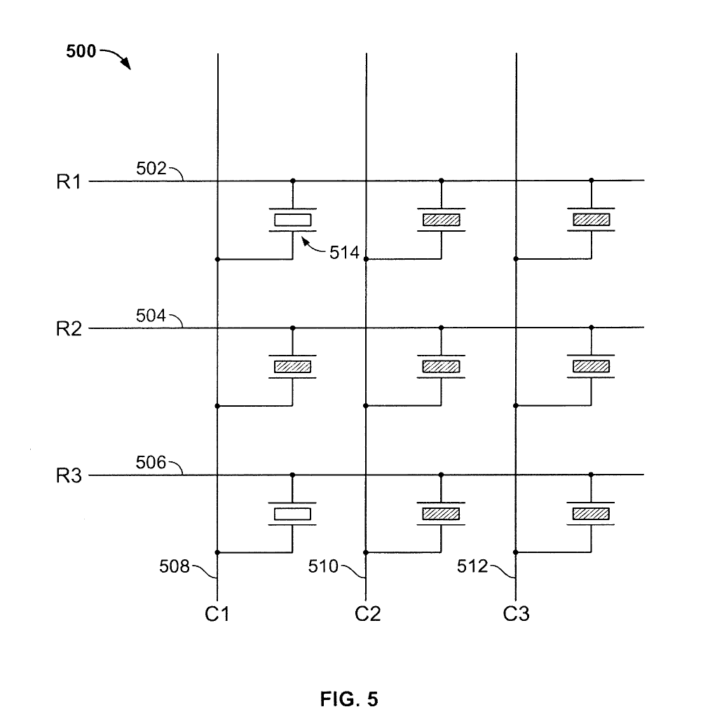

FIG. 5 illustrates a configuration and scenario used in the illustrative examples described in this section. A 3×3 passive matrix EPD array 500 (which may, e.g., be a portion of a larger array) is shown. The Array 500 comprises a plurality of row electrodes 502, 504, and 506, also labeled R1, R2, and R3, respectively, in FIG. 5. The array 500 further comprises a plurality of column electrodes 508, 510, and 512, also labeled as C1, C2, and C3, respectively. Each intersection of a row electrode and a column electrode has associated with it an electrophoretic display element, such as element 514 at the intersection of the first row 502 and first column 508. In the discussion below, a display element such as element 514 may be referred to by a set of Cartesian-style coordinates identifying the corresponding row and column number; e.g., element 514 may be identified as (R1, C1), because it is in row R1 and column C1.

The state of the 3×3 array 500 as shown in FIG. 5 is assumed to be as follows: All nine display elements in the array have been reset to a black/background state in which the white charged pigment particles have been driven to the bottom (non-viewing side) of the display elements; and, considering for present purposes only the elements in the first column, elements (R1, C1) and (R3, C1) are to be switched to a white state (charged pigment particles driven to the top, i.e., viewing, surface) and element (R2, C1) is to retain its initial, black state (particles at the bottom), through the successive scanning of rows R1, R2, and R3. The following paragraphs describe various driving schemes for driving the first column (C1) elements to the end state shown in FIG. 5 from an initial state in which the cells have been reset to all black.

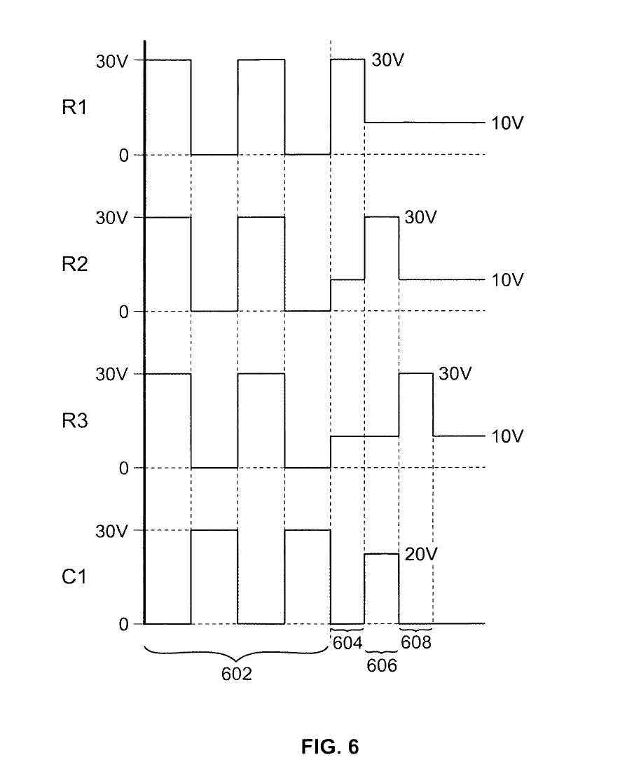

FIG. 6 shows a driving scheme for a basic passive matrix EPD. For a basic passive matrix EPD, the pixels to be switched in the scanning row are under the highest driving energy, which is proportional to the driving voltage Vd times the pulse width (i.e., how long the driving voltage Vd is applied). The non-switching pixels in the scanning row, and the pixels in non-scanning rows, typically are subjected to one third the maximum driving energy (see the discussion in section A above). Therefore, as long as the threshold effect of the EPD cells comprising the pixels is more than one third the maximum driving energy, the cross bias effect will not in theory affect the image quality adversely.

Referring to FIG. 6, the region labeled 602 comprises a reset cycle in which all cells are driven to an initial black/background state in which the charged pigment particles are at the bottom of the cells. As shown in FIG. 6, all three rows are set for a first interval at 30 volts while the column electrodes such as column C1 are held at 0 volts, followed by a substantially equal second interval during which the row electrodes are held at 0 V while the column electrodes are set to 30 V, followed by a repetition of the first and second intervals. In one embodiment, the final interval, in which the column electrodes (at the top, i.e., viewing, surface of the display) are driven to 30 V while the row electrodes are held at 0 V results in the positively charged pigment particles are driven to a position away from the column electrodes and near the row electrodes, i.e., to the bottom of the cells. As noted above, the voltages described in this example and the other examples described herein are illustrative only, and the polarity and magnitude of the voltages used will vary depending on the particular design.

Referring further to FIG. 6, the first row R1 is scanned during a first row scanning interval 604, the second row R2 is scanned during a second row scanning interval 606, and the third row R3 is scanned during a third row scanning interval 608. As shown in FIG. 6, when a row is being scanned it is set to the driving voltage Vd=30V and all the other rows are set to ⅓ Vd=10 V. FIG. 6 shows the voltages that would be applied to column electrode C1 during driving of rows R1 to R3 in order to achieve the end state for the column C1 cells as shown in FIG. 5. Cells (R1, C1) and (R3, C1) are to be driven to the white state (charged particles driven to the top). As such, the column electrode C1 is held at 0 V during the scanning of rows R1 and R3 (intervals 604 and 608), with the result that the magnitude of the potential drop between the top of the cells (R1, C1) and (R3, C1) and the bottom of those cells is the full Vd=30 V, such that the charged particles in those cells are driven during scanning of their respective rows to a new position near the top (i.e., column) electrode on the viewing surface side of the display. (Note that the terms top and bottom are arbitrary. As used herein, “top” refers to the viewing surface of the display. This may be the physical “bottom” of the display element in some designs, such as in a microcup design in which the “bottoms” of the microcups form the viewing surface and the seals “tops” of the cups form the surface opposite the viewing surface.) By contrast, cell (R2, C1) is to retain its initial, black/background state. As such, during scanning of row R2 the column electrode C1 is set to 20 V, so that the potential difference across the cell (R2, C1) is only 10 V, i.e., ⅓ the driving voltage Vd and equal to (i.e., not greater than) the nominal threshold voltage Vth, with the result that the charged particles remain in the initial state to which they were reset during the reset cycle 602.

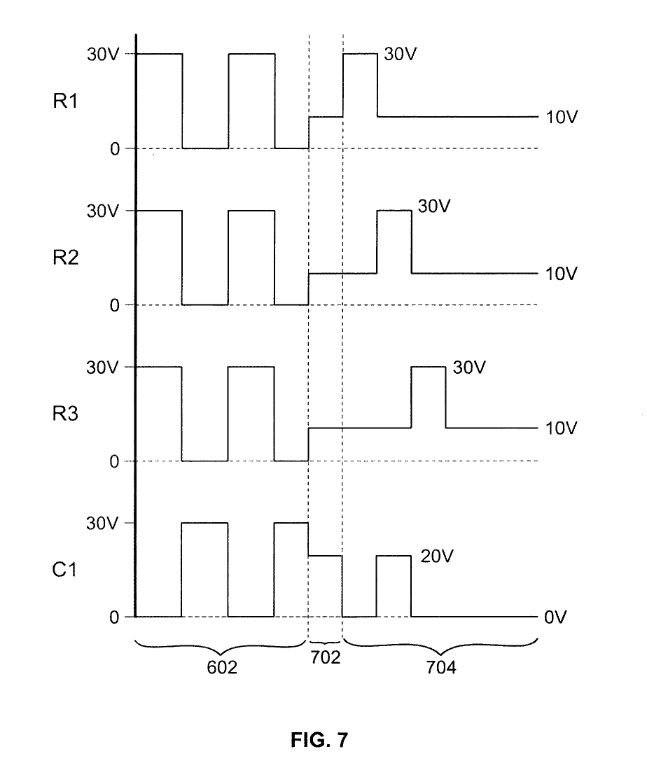

While the passive matrix driving scheme shown in FIG. 6 should work in theory, because it takes into account the mathematically induced relationship between the threshold voltage Vth and the driving voltage Vd as described in Section A above, the scheme shown in FIG. 6 does not address the issue of reverse bias in non-switching pixels. FIG. 7 shows a passive matrix EPD driving scheme in which an intermediate phase has been added to mitigate reverse bias in non-switching pixels. The scheme shown in FIG. 7 starts with the same reset cycle 602 as shown in FIG. 6. An intermediate phase 702 has been added immediately after the reset cycle and immediately before the driving cycle 704, which driving cycle is the same as the intervals 604-608 of FIG. 6. During the intermediate phase 702, the column electrodes such as column electrode C1 are driven to 20 V and the row electrodes are driven to 10 V. Adding such an intermediate phase breaks into two steps the transition from the reset cycle to the driving cycle, thereby mitigating the reverse bias effect. For example, compare the voltages applied to pixel (R2, C1) under the respective schemes shown in FIGS. 6 and 7. Under the FIG. 6 scheme, (R2, C1) is subjected to a 30 V negative bias (column C1 voltage 30 V higher than row R2 voltage) during the final interval of the reset cycle, followed by a positive 10 V bias (voltage at R2=10V, voltage at C1=0 V during interval 604). The net transition is 40 V. Under the FIG. 7 scheme, by comparison, this transition is broken into two steps, a first 20 V transition going from the final interval of the reset cycle to the intermediate phase 702 (going from R2−C1=−30V at the end of the reset cycle to R2−C1=−10V in the intermediate phase) and a second 20 V transition going from the intermediate phase 702 to the first portion of the driving phase 704, which corresponds to interval 604 of FIG. 6 (going from R2−C1=−10V in the intermediate phase to R2−C1=10V in the initial portion of the driving cycle). By breaking this transition into two smaller steps, the reverse bias effect is mitigated.

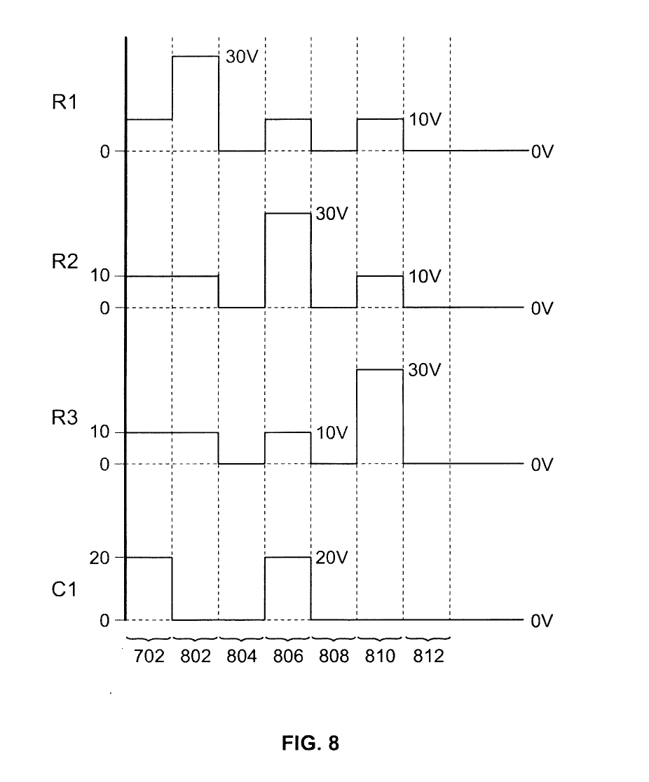

The passive matrix EPD driving scheme shown in FIG. 8 further improves on the scheme shown in FIG. 7. The scheme shown in FIG. 8 commences with the intermediate phase 702 of FIG. 7 and assumes that a reset cycle such as reset cycle 602 (not shown in FIG. 8) has been complete prior to the intermediate phase 702. In the driving cycle following the intermediate phase 702, a “settle” phase has been added after each row is scanned and before the next is scanned. Thus, the first row R1 scanning interval 802 is followed by a settle phase 804 in which all row and column electrodes are set to 0 volts to allow the charged pigment particles to settle and pack together, and to allow the inherent capacitances of the EPD cell structures to discharge, prior to scanning the next row. The second row R2 scanning interval 806 is likewise followed by a settle phase 808, and the third row R3 scanning interval 810 is followed by a settle phase 812. Allowing the inherent capacitances to discharge prior to scanning the next row mitigates the reverse bias effect. Also, introducing a settle phase breaks up DC components applied to the cells, which is beneficial because as noted above applying a DC component without interruption for a long time, even one less than or equal to the nominal threshold voltage Vth, can affect image quality adversely. Finally, in one embodiment the settle phase allows the charged particles to pack together more densely, due to physical, chemical, and/or electrical interactions among the particles and/or between the particles and the dielectric solvent and/or EPD cell structures and materials, enabling the cells to exhibit more fully or strongly the threshold voltage characteristic described herein.

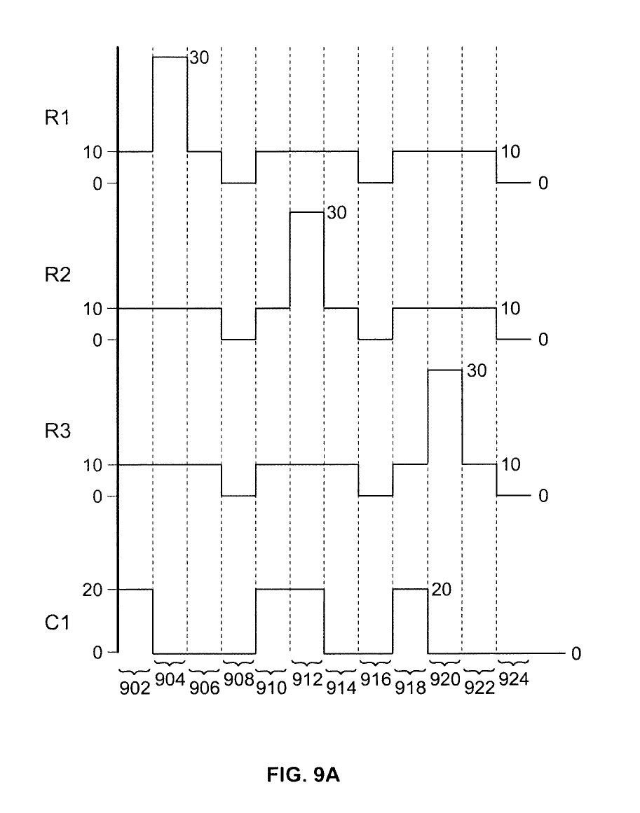

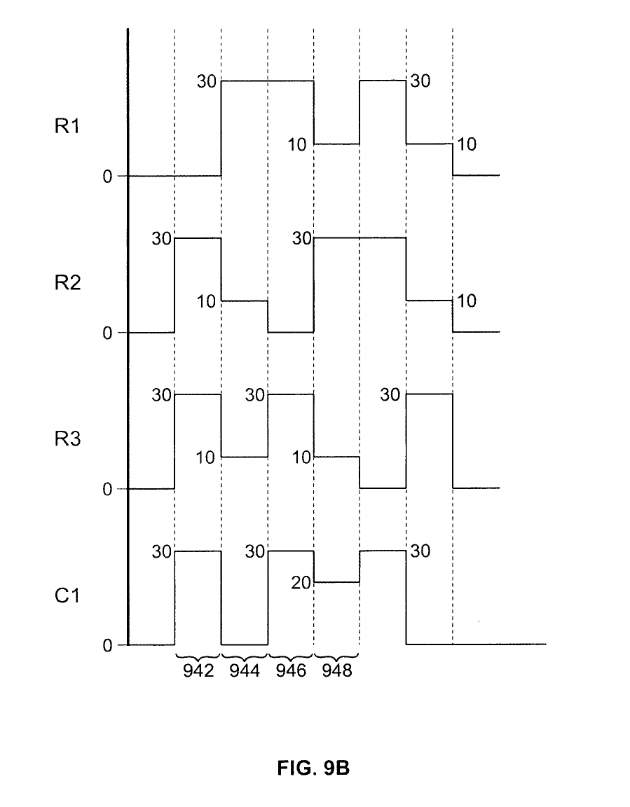

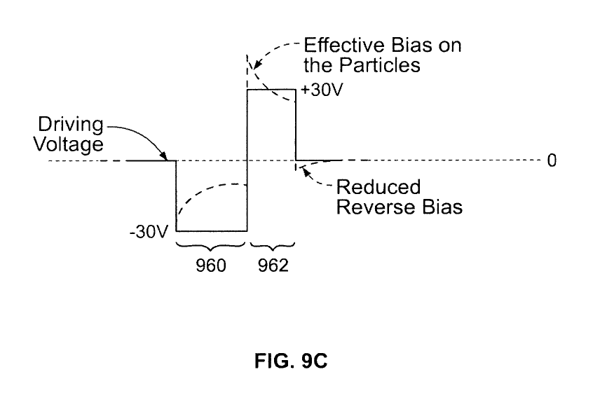

FIG. 9A shows a passive matrix EPD driving scheme in which additional intermediate phases before and after scanning each row have been added to the scheme shown in FIG. 8. An initial intermediate phase of a first type 902 is applied after reset. As shown in FIG. 9A, the first-type intermediate phase 902 is in one embodiment the same as the intermediate phase 702 of FIG. 7 (i.e., columns at 20 V and rows at 10 V). In the scheme shown in FIG. 9A, the first-type intermediate phase 902 is followed by a first row R1 scanning phase 904, which is in turn followed by a second-type intermediate phase 906 (in one embodiment, as shown in FIG. 9, comprising setting the row electrodes to 10 V and the column electrodes to 0 V), followed by a settle phase 908 in which all rows and columns are set to 0 V. The four phase cycle described above for row R1 (phases 902 through 908) is then repeated for the second row R2 (phases 910 through 916) and third row R3 (phases 918 through 924).

In one embodiment, introduction in the scheme shown in FIG. 9A of the additional intermediate phases results in each pixel being subjected first to a negative bias voltage (first-type intermediate phase) and then to a positive bias voltage of equal magnitude but opposite polarity (second-type intermediate phase), in alternating fashion, which reduces particle migration caused by applying the same cross bias voltage for a prolonged period without interruption. As in the scheme shown in FIG. 8, the settle phase allows the particles to settle and pack together. In addition, adding the second-type intermediate phases after scanning reduces the step down in bias voltage that occurs after scanning in a scheme such as that shown in FIG. 8 (i.e., one in which a settle phase is added after scanning), thereby reducing further the effect of induced reverse bias.

FIG. 9B shows a passive matrix EPD driving scheme in which a driving cycle such as that shown in FIG. 6 (intervals 604-608) has been modified to include a pre-drive pulse before each row is scanned. The driving waveforms shown in FIG. 9B and described more fully below use an inverse driving pulse, referred to herein as a pre-drive pulse, to first drive the particles in pixels in the scanning row in the direction of the electrode opposite the one to which the particles in each pixel in the scanning row would be driven during scanning if the data associated with the pixel were such that the driving biasing voltage were to be applied to change the display state of the electrode. After the pre-drive pulse has charged the pixel to reverse polarity, the forward driving pulse is then applied to drive the particles to the designated electrode.

FIG. 9B shows an exemplary driving waveform in which such a pre-drive pulse precedes each scanning cycle. In the example shown in FIG. 9B, it is assumed the pixels contain positively charged white pigment particles suspended in a black dielectric solvent, that the reset state is the black display state in which the charged particles have been driven to a position at or near the row (bottom) electrode, and that the data to be written is such that in the column C1 the pixels in rows R1 and R3 are to be written to the white display state (particles at or near the column (top) electrode C1) and the pixel in row R2 is to retain the black display state. When a row (e.g., R1) is addressed, the pixels in the scanning row are first reset to the black display state during a pre-drive phase 942, during which the row to be scanned next, i.e., row R1, is set to 0V and the non-scanning rows R2 and R3 and column electrodes such as column electrode C1 are set to 30V, resulting in an inverse driving (i.e., reset) bias condition being applied to the pixels in row R1 and no bias being applied to pixels in non-scanning rows. Row R1 is then set to 30V during a row R1 scanning phase 944. During row R1 scanning phase 944, column electrode C1 is set to 0V to cause the associated pixel in column C1 row R1 to be driven to the white display state, in accordance with the display data associated with that pixel. During row R1 scanning phase 944, non-scanning rows R2 and R3 are set to 10V to avoid changing the display state of pixels in such non-scanning rows as a result of cross bias. Scanning of row R1 is followed by a pre-drive phase 946 for row R2, in which row R2 is set at 0V and rows R1 and R3 and column electrodes such as C1 are set to 30V, such that an inverse driving bias condition is applied to the pixels of row R2, driving them to the black display state, while zero bias is applied to pixels in non-scanning rows. During row R2 scanning phase 948, row electrode R2 is set to 30V, row electrodes R1 and R3 are set to 10V to maintain the display state of pixels in the non-scanning rows, and column electrode C1 is set to 20V to cause the pixel associated with row R2 and column C1 to retain its black display state (in accordance with the scenario described above). Row R3 pre-drive phase 950 and scanning phase 952 are similar to the corresponding phases 942 and 944 for row R1 and result in the pixel associated with row R3 and column C1 being driven to the white display state.

FIG. 9C illustrates the reduced reverse bias that can be achieved by including a pre-drive phase such as shown in FIG. 9B. The driving voltage (bias) applied to a pixel during a pre-drive phase 960 and a driving phase 962 are shown as a solid line, and the effective bias on the charged particles of the pixel as a dotted line. The reverse bias effect during transition is reduced due to two factors. First, the reverse charge on the pixel cancels some of the reverse bias. Second, the voltage at the transition is higher (the bias is −30V during the pre-drive phase and swings to +30V during driving) and therefore drives and packs the particles tighter, resulting in the particles being impacted by the reverse bias effect to a lesser degree.

C. Improving Performance by Driving Background Areas to Background Color to Display an Image

In certain passive matrix EPDs, the time required to drive charged particles from the bottom of the EPD cells to the top (viewing side) of the cells may be longer than the time required to drive the charged particles in the opposite direction (i.e., from top to bottom). For example, in an EPD comprising microcup electrophoretic display cells, in certain embodiments the time to drive the charged pigment particles to the non-viewing side of the microcups may be less than the time required to drive the charged pigment particles from the non-viewing side to the viewing side for one or more of a number of possible reasons, including without limitation the shape of the microcups, the characteristics of the dielectric solvent and/or charged pigment particles and/or dynamics between them, and/or the materials used to form one or more structures associated with the microcup.

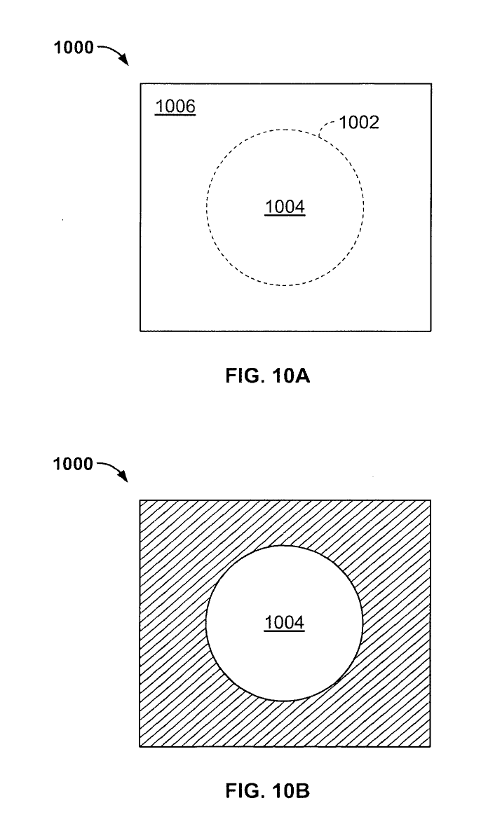

FIGS. 10A and 10B illustrate an approach used in one embodiment to display a desired image on a passive matrix EPD in which charged particles can be driven away from the viewing surface more quickly than they can be driven from the non-viewing side to the viewing surface. FIG. 10A shows a passive matrix electrophoretic display 1000 on which an image of a circle is to be displayed, as indicated by the dashed line 1002 in the center of the display 1000, which defines an image area 1004 inside the dashed line 1002 and a background area 1006 outside the circle, e.g., in accordance with image data provided to the display 1000 and/or associated circuitry and/or processing elements. The typical approach to displaying such an image has been to first reset all pixels to the black/background state (charged particles to the non-viewing side of the cells) and then drive the cells in the image area, such as image area 1004 of FIG. 10A to the white state by driving the charged particles in such cells to the viewing surface.

FIG. 10A shows a starting point in which, instead of driving all pixels to the black/background color state, all pixels have been driven to an initial state in which the charged pigment particles are at the viewing surface (sometimes referred to as the “on” state). From this state, the cells in the background area 1006 are driven to the black/background state by driving the charged pigment particles in such cells away from the viewing surface, leaving in the image area 1004 an image in white of the circle defined by dashed line 1002, as shown in FIG. 10B.

While the illustrative example shown in FIGS. 10A and 10B describes white charged pigment particles and a solvent having a black or other background color, the same technique may be used in displays in which pigment particles and/or solvents of different and/or multiple colors are used, such as to provide a color display. Indeed, the technique may be applied advantageously in any EPD in which it takes less time to drive charged particles “down” (i.e., to the non-viewing surface of the display) than “up” (i.e., from the bottom or non-viewing surface to the top or viewing surface).

D. Using an In-line Resistor and/or Short Pulse Width to Reduce the Effect of Induced Reverse Bias

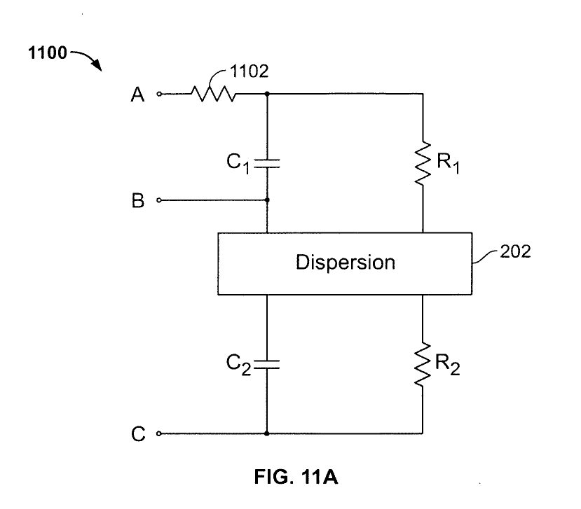

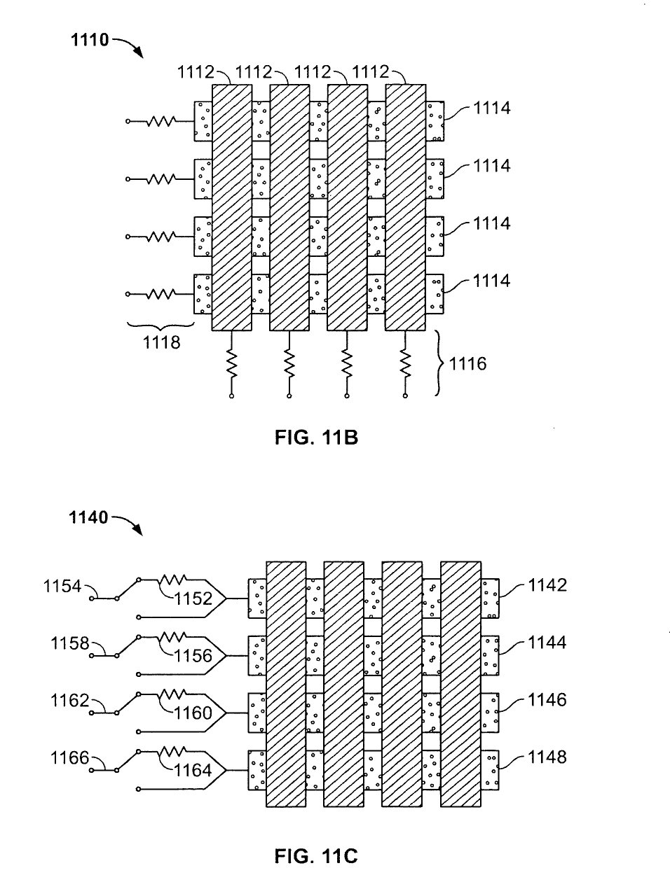

The passive matrix EPD driving schemes described in Section B above reduce the effect of induced reverse bias by breaking large voltage transitions into two steps and/or by including a settle phase during which the capacitances that cause the induced reverse bias effect may discharge. It has been found that adding an inline (serial) resistor prior to the upper layer or lower layer insulating structures, as applicable (i.e., prior to the capacitance and resistance associated with those structures), reduces further the induced reverse bias effect by not allowing the capacitances associated with the cell to become fully charged prior to the next voltage transition. FIG. 11A shows an equivalent circuit 1100 for an EPD cell to which such an inline resistor has been added.

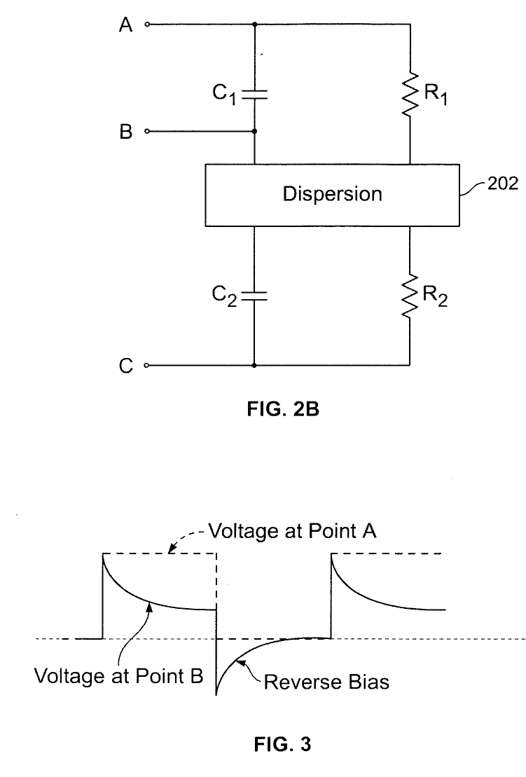

Comparing FIG. 11A with FIG. 2B, the equivalent circuits are the same except that in FIG. 11A an inline resistor 1102 has been added prior to the capacitance and resistance associated with the upper insulating structures of the EPD cell. In one embodiment, each row electrode, each column electrode, or both, is connected to the associated driver circuit via the inline resistor. In one embodiment, the inline resistor comprises a discrete component applied on the EPD electrode substrate, or on the connector/adaptor described above, or on the driver IC circuit board. In one embodiment, the inline resistors may be implemented in the driver IC, e.g., as a thick or thin film resistor.

FIG. 11B shows a 4×4 array (or portion of an array) in which an inline resistor has been added between the row and column electrodes and their respective drivers. The array 1110 comprises a plurality of column electrodes 1112 and a plurality of row electrodes 1114. Each of the plurality of column electrodes 1112 is connected via a corresponding one of a plurality of column electrode inline resistors 1116 to its associated column driver (not shown). Likewise, each of the plurality of row electrodes 1114 is connected via a corresponding one of a plurality of row electrode inline resistors 1118 to its associated row driver (not shown). As noted above, in alternative embodiments just the row or just the column electrodes may be connected to their respective drivers via an inline resistor.

FIG. 11C shows an alternative arrangement used in one embodiment, in which a switch is provided to enable the inline resistor to be removed from the circuit during driving. FIG. 11C shows an array 1140 comprising row electrodes 1142, 1144, 1146, and 1148. Row electrode 1142 has associated with it an inline resistor 1152 and a switch 1154. Row electrode 1144 has associated with it an inline resistor 1156 and a switch 1158. Row electrode 1146 has associated with it an inline resistor 1160 and a switch 1162. Row electrode 1148 has associated with it an inline resistor 1164 and a switch 1166. Each of the switches 1154, 1158, 1162, and 1166 has two positions, a first position in which the associated inline resistor is included in the path from the driver to the electrode and a second position in which the inline resistor is bypassed. The switches 1154, 1162, and 1166 are shown in the first position and switch 1158 is shown in the second position. In one embodiment, the switch associated with a row electrode is placed in the second (i.e., bypass) position during driving of the associated row, with the result that the inline resistor is not included in the path from the driver to the electrode, such that the resistor is not present to affect adversely (i.e., reduce) the bias voltage applied across the electrophoretic dispersion (i.e., by virtue of the voltage drop that would occur across the inline resistor if it were included in the circuit). In one embodiment, when scanning of a particular row is completed, the switch associated with that row changes from the second position to the first position, thereby re-inserting the inline resistor into the path from the driver to the electrode. This configuration enables the benefit of using an inline resistor to reduce reverse bias to be realized without having to suffer the degradation of performance that might otherwise be caused by including the inline resistor when the associated electrode is being driven. Of course, this configuration may be used as well (or instead) with column electrodes, depending on the design of a particular passive matrix EPD.

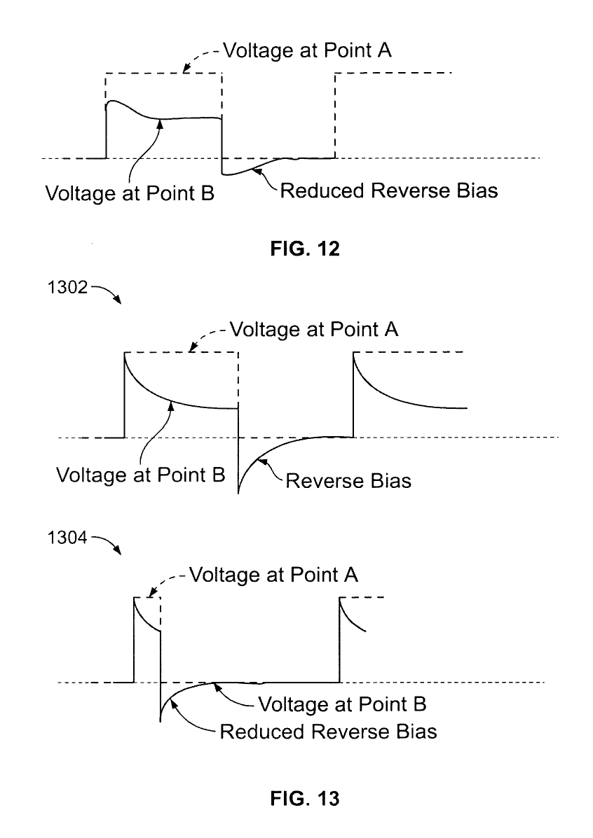

FIG. 12 plots voltage versus time for points A and B of FIG. 11A, which correspond to points A and B in the EPD cell shown in FIG. 2A. Comparing FIG. 12 with FIG. 3, one can see that adding the inline resistor 1102 slows the charging of the capacitances C1 and C2 of the equivalent circuit shown in FIG. 11A, resulting in a reduced reverse bias effect. Because of this added inline resistor, the effective bias on the electrophoretic dispersion is also reduced, as a result of the voltage drop across the inline resistor. Therefore an optimization is required to select the inline resistor value that is high enough to reduce the reverse bias but also low enough to keep the effective bias at an acceptable level. The resistance value of the inline resistor depends on the pixel size of the display and the number of pixels on the same row or column. The electrical characteristics of the dispersion and the insulator layers also affect the selection of the resistance of the inline resistor. In one embodiment, it is in the mega-ohm range.

Yet another measure that may be taken to reduce the reverse bias effect by preventing the inherent capacitances of the EPD cell structures from charging fully is to reduce the pulse width used for driving. FIG. 13 illustrates the reduction in reverse bias that is achieved by using a shorter pulse width. The upper voltage versus time plot 1302 is a reproduction of the plot shown in FIG. 3. The lower voltage versus time plot 1304 illustrates the effect of using a shorter pulse width, which is to reduce the reverse bias effect by not allowing the capacitances associated with cell structures, such as the capacitances C1 and C2 of FIG. 11A, to become fully charged prior to the next voltage transition. In one embodiment, it may be necessary to apply an increased number of the shorter cycles in order to drive to a new state the EPD cells that are to be switched in accordance with the image data. The pulse width must be long enough to at least partially induce the particles to move in the desired direction, but also short enough to reduce the reverse bias. Therefore the optimization of the pulse width depends in one embodiment on factors such as the particle mobility and the EPD electrical characteristics.

E. Adding a Balance Phase to Restore Cells in Non-Scanning Rows to the Same Initial State for Scanning

As noted above, one of the factors that can affect the actual threshold voltage of an EPD cell under a given set of conditions is the initial state of the EPD cell, and in particular the state of the charged pigment particles within the cell. For example, if the charged pigment particles are well settled and packed together densely at the bottom of the cell, exposing the color of the dielectric solvent, the actual threshold voltage will be greater than if the charged pigment particles are not well-settled and densely packed. Under the latter conditions, the voltage required to cause at least some of the charged particles to move towards the upper (viewing) surface may be less than that required under the former circumstances.

The cross bias effect can cause some cells in a row to transition to a different initial state than other cells in that same row prior to the scanning of said row. As described above, while other rows are scanned, voltages are applied to selected column electrodes to cause the respective scanning row cells associated with such selected column electrodes to either change or retain their state, depending on the design. These voltages can cause the cells in non-scanning rows that happen to be in the same columns to change their initial state to a degree, even though the voltage applied to such cells is at or below the nominal threshold voltage for the cell. That is, even if the cross bias voltage is less than the nominal threshold voltage, the cells subjected to such a cross bias voltage may experience some change in their initial state. For example, in an embodiment in which all cells are reset to a black/background state (charged pigment particles on the bottom or non-viewing side of the cells) prior to scanning, the charged pigment particles in cells subjected to cross bias in non-scanning rows might become less densely packed, and some particles might begin to migrate towards the viewing surface. During subsequent scanning, such variations in the initial state may result in undesired variation in the response to the driving voltages applied during scanning, which may result in a non-uniform image.

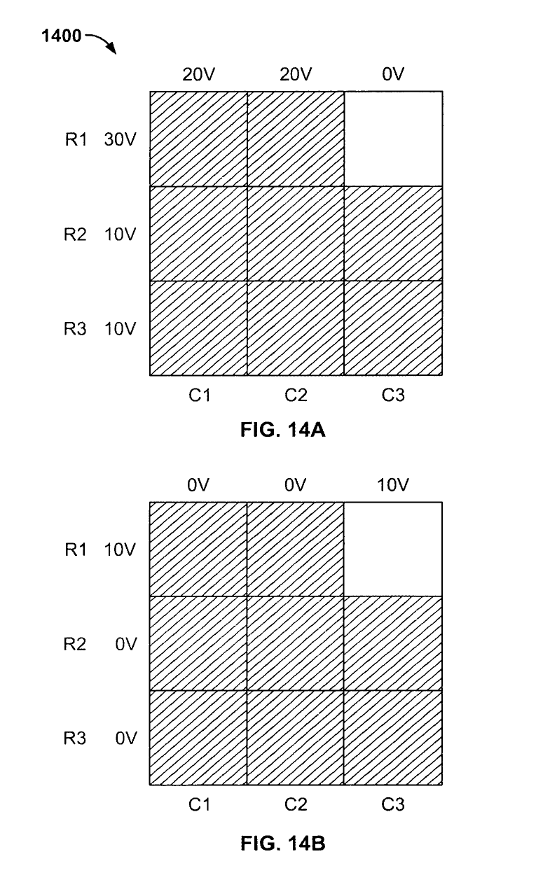

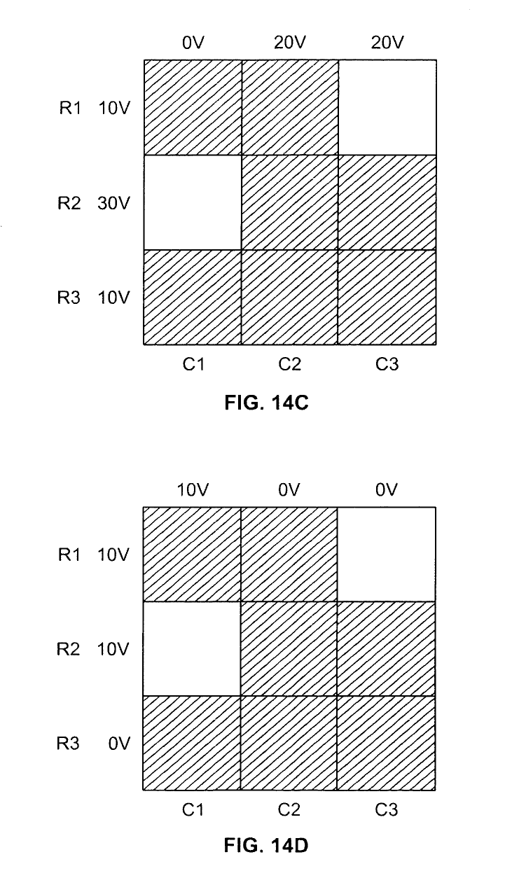

Use of a balance phase to restore cells in non-scanning rows to the same initial state is disclosed. FIG. 14A shows an exemplary passive matrix EPD comprising a 3×3 array of EPD cells (or pixels comprising one or more EPD cells) during scanning of the first row R1. In the example shown in FIG. 14A, the first and second cells of the row, i.e., cells (R1, C1) and (R1, C2), are being maintained at the initial black/background state (to which it is assumed in the example all cells have been reset prior to scanning) by applying 20 V to the column electrodes for columns C1 and C2 while the scanning voltage of Vd=30 V is applied to row R1. The third cell in row R1, cell (R1, C3), is being driven to the white state by holding the column electrode for column C3 at 0 V during scanning, such that the driving voltage Vd=30 V is applied across the cell. During scanning of row R1 as shown in FIG. 14A, the cells in the third column C3 in the non-scanning rows—i.e., cells (R2, C3) and (R3, C3)—are subjected to a positive cross bias of 10 V (lower row electrode voltage greater than upper column electrode by 10 V). This cross bias, while assumed to be below the nominal threshold voltage of the cells, may as noted above be sufficient to cause at least some particles in these cells to migrate in the direction of the viewing surface, or at least to become less densely packed together. Note that the remaining cells of the non-scanning row are subjected to a 10 V negative cross bias (upper/column electrode voltage greater than lower/row electrode voltage by 10 V), which does not have to be offset by the balance phase described below because it tends to keep the charged particles in the position at the bottom of the EPD cells to which they have been reset.

FIG. 14B illustrates a balance phase used in one embodiment to return cells in non-scanning rows to the same initial state for scanning. In the balance phase shown in FIG. 14B a negative bias voltage is applied to cells subjected to a positive cross bias during scanning of row R1. As shown in FIG. 14B, in one embodiment this accomplished by setting the rows that were non-scanning rows during the scanning of row R1, i.e., rows R2 and R3, to 0 V while applying 10 V to the column electrodes for columns which were set to 0 V during the scanning of row R1 (i.e., columns associated with cells in row R1 that were switched from black/background to white during scanning of row R1). Columns not associated with cells that were switched on during the scanning of row R1, in this case columns C1 and C2, are set to 0 V, with the result that no bias is applied to the cells of rows R2 and R3 that were not affected by a positive cross bias during scanning of row R1—i.e., cells (R2, C1), (R2, C2), (R3, C1), and (R3, C2). The previously-scanned row R1 is set to 10 V, to maintain the image quality by ensuring that the cells of that row that were switched on during scanning remain fully on (i.e., particles at the viewing surface) by ensuring that no negative cross bias is applied to those cells. The resulting 10 V positive bias applied to the non-switched cells of row R1 is less than or equal to the threshold voltage Vth and is not applied long enough to affect image quality adversely. In one embodiment, these cells will be reset fully to the black/background state, along with all the other cells, during a reset cycle prior to the next scanning cycle.

FIG. 14C illustrates the scanning of the second row R2. In the example shown, the first cell of the row, (R2, C1), is switched to the white state by applying 0 V to the associated column C1 while the driving voltage Vd=30 V is applied to row R2. The remaining cells of the row R2 are maintained in the black/background state by applying 20 V to columns C2 and C3. In the scanning cycle for row R2 shown in FIG. 14C, the first cell in row R3—i.e., cell (R3, C1)—is subjected to a 10 V positive cross bias. FIG. 14D illustrates a balance phase used in one embodiment to counteract the effect of the positive cross bias on cells in the non-scanning row R3. As in FIG. 14B, the non-scanning row R3 is set to 0 V while the columns associated with cells subjected to the positive cross bias during scanning of row R2, in this case column C1, are set to 10 V, with the remaining columns being set to 0 V. This results in a negative bias being applied to cell (R3, C1), resetting that cell to the same initial state as cells (R3, C2) and (R3, C3) prior to scanning of row R3. In one embodiment, as shown in FIG. 14D, the effect of positive cross bias on previously-scanned rows, such as row R1, is not counteracted by the balance phase shown in FIG. 14D, because that row has already been scanned and the cells of that row will be reset to a common initial state during a reset cycle that will occur once all rows have been scanned and the display system is ready to display the next frame of image data. In one embodiment, as shown in FIG. 14D, all previously-scanned rows are set to 10 V, to ensure that cells in those rows are maintained either at 0 V bias or 10 V positive bias, to maintain image quality by keeping charged particles in cells previously switched to the white or “on” state from migrating away from the viewing surface during the balance phase.

Although the foregoing invention has been described in some detail for purposes of clarity of understanding, it will be apparent that certain changes and modifications may be practiced within the scope of the appended claims. It should be noted that there are many alternative ways of implementing both the process and apparatus of the present invention. Accordingly, the present embodiments are to be considered as illustrative and not restrictive, and the invention is not to be limited to the details given herein, but may be modified within the scope and equivalents of the appended claims.

What is claimed is:

1. A method for mitigating the effect of induced reverse bias in a passive matrix electrophoretic display comprising performing an intermediate biasing phase prior to the driving cycle, the biasing conditions of the intermediate biasing phase being selected so as to break into at least two steps the transition from the bias condition present prior to the driving cycle to the bias condition applied during the driving cycle.

2. A method for mitigating the effect of induced reverse bias in a passive matrix electrophoretic display comprising an array of electrophoretic display elements, the method comprising:
resetting the electrophoretic display elements to a first stable state by applying a reset biasing voltage across the display elements;
applying an intermediate biasing voltage across the display elements; and
applying a driving biasing voltage across at least one selected display element to drive said at least one selected display element to a second stable state.

3. The method of claim 2, wherein:
at least one display element not selected to be driven to the second stable state is subjected to cross bias as a result of said step of applying a driving biasing voltage across at least one selected display element; and
the intermediate biasing voltage has a value between the reset biasing voltage and the cross bias voltage;
whereby the induced reverse bias effect is mitigated with respect to said at least one display element not selected to be driven to the second stable state by breaking the transition from application of the reset biasing voltage to application of the cross bias into at least two steps.

4. The method of claim 2, wherein the reset biasing voltage comprises the final step in a reset cycle, the reset cycle comprising applying to the array of electrophoretic display elements a series of biasing voltages of alternating polarity, whereby the electrophoretic display elements are set to the first stable state.

5. A method for displaying an image on a passive matrix electrophoretic display, the display comprising a plurality of electrophoretic display elements arranged in a plurality of rows and configured to display an image by scanning said plurality of electrophoretic display elements row by row, the method comprising interposing a settle phase subsequent to the driving phase for each scanned row and prior to the driving phase of the next row to be scanned, the settle phase comprising subjecting the plurality of electrophoretic display elements to approximately zero bias.

6. A method for displaying an image on a passive matrix electrophoretic display, the display comprising an array of electrophoretic display elements arranged in a plurality of rows, the method comprising:
resetting said plurality of electrophoretic display elements to a first stable state; and
setting selected ones of said plurality of electrophoretic display elements to a second stable state, said step of setting selected ones of said plurality of electrophoretic display elements to a second stable state comprising scanning said plurality of electrophoretic display elements row by row, said scanning comprising applying a driving voltage to an electrode associated with the row being scanned; and
applying to said electrode associated with the row being scanned, for an interval after each row is scanned and prior to commencing scanning of the next row to be scanned, a settle phase voltage selected so as to ensure that electrophoretic display elements associated with said electrode are not subjected to any substantial positive or negative bias during the interval during which the settle phase voltage is being applied.

7. The method of claim 6, wherein the settle phase voltage is zero volts.

8. The method of claim 6, further comprising applying a first intermediate phase subsequent to the scanning phase and prior to the settle phase, said first intermediate phase comprising:
applying a first intermediate biasing voltage to said array of electrophoretic display elements;
wherein said first intermediate biasing voltage is of the same polarity as said driving voltage and has a magnitude that is less than said driving voltage and greater than zero;
whereby the transition from the driving voltage to the settle phase is broken into at least two steps.

9. The method of claim 8, further comprising applying a second intermediate phase subsequent to the settle phase and prior to scanning of the next row to be scanned, said second intermediate phase comprising:
applying a second intermediate biasing voltage to said array of electrophoretic display elements;
wherein said second intermediate biasing voltage is of the opposite polarity as said first intermediate biasing voltage.

10. The method of claim 9, wherein said second intermediate biasing voltage is of the same magnitude but of the opposite polarity as said first intermediate biasing voltage.

11. A method for mitigating the effect of induced reverse bias in a passive matrix electrophoretic display comprising performing prior to a driving biasing phase for each scanning row a pre-drive biasing phase during which a pre-drive biasing voltage having a polarity opposite the polarity of a driving biasing voltage applied to selected pixels of the scanning row during the driving biasing phase for the scanning row to change the display state of said selected pixels from a first display state to a second display state.

12. The method of claim 11 wherein the pre-drive biasing voltage is equal in magnitude but opposite in polarity to the driving biasing voltage.

13. The method of claim 11 further comprising applying to a row electrode associated with a non-scanning row during the pre-drive phase of a scanning row other than the non-scanning row a voltage that results in zero bias being applied to the pixels of the non-scanning row.

14. A passive matrix electrophoretic display system comprising:
an array of electrophoretic display elements; and
driving circuitry configured to:
reset the electrophoretic display elements to a first stable state by applying a reset biasing voltage across the display elements;
apply an intermediate biasing voltage across the display elements; and
apply a driving biasing voltage across at least one selected display element to drive said at least one selected display element to a second stable state.

15. The system of claim 14, wherein:
at least one display element not selected to be driven to the second stable state is subjected to cross bias as a result of applying the driving biasing voltage across at least one selected display element; and
the intermediate biasing voltage has a value between the reset biasing voltage and the cross bias voltage;
whereby the induced reverse bias effect is mitigated with respect to said at least one display element not selected to be driven to the second stable state by breaking the transition from application of the reset biasing voltage to application of the cross bias into at least two steps.

16. A passive matrix electrophoretic display system, comprising:
an array of electrophoretic display elements positioned between a first electrode layer comprising a plurality of column electrodes and a second electrode layer comprising a plurality of row electrodes; and
driving circuitry configured to:
reset said plurality of electrophoretic display elements to a first stable state; and
set selected ones of said plurality of electrophoretic display elements to a second stable state by a method comprising:
scanning said plurality of electrophoretic display elements row by row; and
applying to the row and column electrodes, for an interval after each row is scanned and prior to commencing scanning of the next row to be scanned, a settle phase voltage selected so as to ensure that the array of electrophoretic cells are not subjected to any positive or negative bias during the interval during which the settle phase voltage is being applied.

17. A passive matrix electrophoretic display system, comprising:
an array of electrophoretic display elements positioned between a first electrode layer comprising a first plurality of electrodes and a second electrode layer comprising a second plurality of electrodes; and
driving circuitry, comprising for each electrode of said first electrode layer:
a driver configured to apply a driving voltage to the electrode; and
a serial resistor positioned between the driver and the electrode.

18. The system of claim 17, wherein the electrodes of said first electrode layer comprise row electrodes.

19. The system of claim 17, wherein the electrodes of said first electrode layer comprise column electrodes.

20. The system of claim 17, wherein said first electrode layer comprises a plurality of row electrodes and said second electrode layer comprises a plurality of column electrodes, and wherein said driving circuitry further comprises, for each electrode of said second layer of electrodes:
a driver configured to apply a driving voltage to the electrode; and
a serial resistor positioned between the driver and the electrode.

21. The system of claim 17 wherein each of said electrophoretic display elements has a display element resistance and a display element capacitance associated with it and the serial resistor has a resistance that is selected based at least in part on said display element resistance and said display element capacitance.

22. The system of claim 17, wherein:
said first electrode layer comprises a plurality of row electrodes;
said electrophoretic display is configured to display an image by a method comprising scanning said electrophoretic display elements row by row and driving selected ones of said electrophoretic display elements from a first stable state to a second stable state though application of a driving voltage to the associated row electrode during a driving interval;
said electrophoretic display elements may be subjected to an induced reverse bias effect during the transition from application of the driving voltage during the driving interval to application of the voltage applied after the driving interval; and
the presence of said serial resistor mitigates said induced reverse bias effect by preventing said display element capacitance from charging fully during the driving interval.

23. The system of claim 17, further comprising for each electrode of said first electrode layer a switch configured to bypass said serial resistor during a first interval during which the driving voltage is applied to the electrode and to not bypass the serial resistor during a second interval during which a voltage other than the driving voltage is being applied to the electrode.

24. A passive matrix electrophoretic display system comprising:
an array of electrophoretic display elements; and
driving circuitry configured to apply a driving biasing voltage across at least one selected display element to drive said at least one selected display element from a first stable state to a second stable state by a method comprising applying a driving voltage to an electrode associated with said at least one selected display element for a driving interval corresponding to a driving pulse width;
wherein each of said electrophoretic display elements has a display element capacitance associated with it and said driving pulse width is selected so as to ensure that the display element capacitance does not charge fully during the driving interval;
whereby the induced reverse bias effect is mitigated.

25. A passive matrix electrophoretic display system comprising:
an array of electrophoretic display elements arranged in a plurality of rows; and
driving circuitry configured to:
scan said array of electrophoretic display elements row by row by applying a driving voltage to an electrode associated with the row being scanned; and
apply a balance phase to at least one non-scanning row subsequent to the scanning of a scanned row, the balance phase comprising subjecting
electrophoretic display elements associated with said at least one non-scanning row to a balancing bias voltage, wherein the balancing bias voltage tends to place said electrophoretic display elements associated with said at least one non-scanning row in the state they were in prior to the scanning of the scanned row.

26. A passive matrix electrophoretic display system comprising:
an array of electrophoretic display elements arranged in a plurality of rows; and
driving circuitry configured to:
reset the electrophoretic display elements to a first stable state by applying a reset biasing voltage having a first polarity; and
scan said array of electrophoretic display elements row by row by applying a driving voltage to an electrode associated with the row being scanned, whereby a driving bias voltage of a second polarity opposite of the first polarity is applied to the display elements associated with the row being scanned that are to be driven to the second stable state;
wherein at least one display element in a non-scanning row is subjected to a cross bias of the second polarity during scanning of a scanning row, the cross bias tending to place said at least one display element in a non-scanning row at least in part in a state other than the first stable state; and
wherein the driving circuitry is further configured to apply a balance phase subsequent to scanning each row and prior to scanning the next row to be scanned, the balance phase comprising applying to said at least one display element in a non-scanning row that was subjected to a cross bias a balance phase bias of the first polarity that is equal to or greater than in magnitude than the cross bias;
whereby said at least one display element in a non-scanning row that was subjected to a cross bias is reset to the same state as display elements of the same row that were not subjected to the cross bias of the second polarity.

27. The system of claim 26, wherein the balance phase is applied only to rows that have not yet been scanned.

28. The system of claim 26, further comprising logic configured to determine which display elements of non-scanning rows are subjected to the cross bias of the second polarity and to apply said balance phase bias to said display elements of non-scanning rows subjected to the cross bias of the second polarity.

29. A method for causing an image to be displayed on a passive matrix electrophoretic display comprising an array of electrophoretic display elements arranged in a plurality of rows, the method comprising:
scanning said array of electrophoretic display elements row by row, said scanning comprising applying a driving voltage to an electrode associated with the row being scanned; and
applying to at least one non-scanning row, subsequent to scanning a scanned row, a balance phase comprising applying a balance biasing voltage to counteract the effect of cross bias on at least one display element in said non-scanning row.

30. A method for causing an image to be displayed on a passive matrix electrophoretic display comprising an array of electrophoretic display elements arranged in a plurality of rows, the method comprising:
resetting said plurality of electrophoretic display elements to a first stable state;
scanning said array of electrophoretic display elements row by row, said scanning comprising setting selected display elements of the scanned row to a second stable state; and
applying a balance phase subsequent to scanning each row and prior to scanning the next row to be scanned, wherein during the balance phase a balance biasing voltage is applied to counteract the effect of cross bias on at least one display element in a non-scanning row that was subjected to a cross bias that tended to drive it to a state other than the first stable state;
whereby said at least one display element in a non-scanning row that was subjected to cross bias is restored to the first stable state.

31. A method for displaying an image on a passive matrix electrophoretic display, the display comprising a plurality of display elements, each display element having a viewing surface side and a non-viewing surface side and each comprising a quantity of an electrophoretic dispersion comprising a plurality of charged pigment particles dispersed in a colored dielectric solvent, the method comprising:
driving the plurality of display elements to a first stable state in which the charged pigment particles of each display element are in a position at or near the viewing surface side of the display element; and
driving to a second stable state display elements located in portions of the display in which the image is not to be displayed, the second stable state comprising a state in which the charged pigment particles of each display element driven to the second stable state are in a position at or near the non-viewing surface side of the display element;
whereby the image is displayed in the color of the charged pigment particles in portions of the display in which the display elements have been left in the first stable state and a contrasting background color is displayed in portions of the display in which the display elements have been driven to the second stable state.

32. The method of claim 31, wherein the display requires less time to drive display elements from the first stable state to the second stable state than to drive display elements from the second stable state to the first stable state.

33. A passive matrix electrophoretic display system, comprising:
a plurality of display elements, each display element having a viewing surface side and a non-viewing surface side and each comprising a quantity of an electrophoretic dispersion comprising a plurality of charged pigment particles dispersed in a colored dielectric solvent; and
a driving circuit configured to:
drive the plurality of display elements to a first stable state in which the charged pigment particles of each display element are in a position at or near the viewing surface side of the display element; and
drive to a second stable state display elements located in portions of the display in which the image is not to be displayed, the second stable state comprising a state in which the charged pigment particles of each display element driven to the second stable state are in a position at or near the non-viewing surface side of the display element;
whereby the image is displayed in the color of the charged pigment particles in portions of the display in which the display elements have been left in the first stable state and a contrasting background color is displayed in portions of the display in which the display elements have been driven to the second stable state.

34. The system of claim 33, wherein the display requires less time to drive display elements from the first stable state to the second stable state than to drive display elements from the second stable state to the first stable state.
